(19) United States
(12) Patent Application Publication
(10) Pub. No.: US 2023/0221300 A1
(43) Pub. Date: Jul. 13, 2023

(54) METHODS AND DEVICES FOR CELL BASED ASSAYS

(71) Applicants: Hoffmann-La Roche Inc., Little Falls, NJ (US); NCL New Concept Lab GmbH, Moehlin (CH)

(72) Inventors: Stephen FOWLER, Rheinfelden (CH); Na Hong QIU, Moehlin (CH); Guojun CHEN, Moehlin (CH)

(73) Assignees: Hoffmann-La Roche Inc., Little Falls, NJ (US); NCL New Concept Lab GmbH, Moehlin, NJ (CH)

(21) Appl. No.: 17/933,447

(22) Filed: Sep. 19, 2022

Related U.S. Application Data
(63) Continuation of application No. PCT/EP2021/057179, filed on Mar. 22, 2021.

(30) Foreign Application Priority Data
Mar. 23, 2020 (EP) 20164950.6

(51) Int. Cl. G01N 33/50; C12M 1/26; C12M 1/32; C12M 1/12; C12M 1/00; C12M 1/34

(52) U.S. Cl. CPC G01N 33/5038; C12M 33/04; C12M 23/12; C12M 25/14; C12M 23/20; C12M 41/46

(57) ABSTRACT

The present invention provides cell assay systems and its use in cell based assays.

Cell based assays have been used broadly in the fields of cell biology, medical science, pharmacology, toxicology, and so on. They are usually performed in a multi-well cell culture plate. The cells can be in the form of suspension, 2D monolayer, or 3D organoids/spheroids according to the assay. In order to meet some special needs, many new systems have been introduced. These include microfluidic cell culture device, 3D membrane based perfusion bioreactor, 3D-cell culture pillar system, and so on. Though bringing some advantages, these systems show more or less drawbacks in terms of complexity, robustness, flexibility, easy handling and cost effectiveness. For example the flow channel of the microfluidic system is prone to be blocked by air bubbles, hydrogel and cell clusters. The 3D-cell culture pillar system is only suitable to prepare multi-spheroids from proliferating cells because it has to use hydrogel to immobilize cells to the pillar. Non-proliferating cells like human hepatocytes cannot form spheroids in the hydrogel. The use of hydrogel also limits the applications of this pillar system as the hydrogel can have impacts on, especially, large molecule diffusion and the assay results. Besides, to immobilize the cells to the tiny pillar can be challenge since the hydrogel need to be solidified in a very short time to avoid evaporation of the small volume.

In view of the shortcomings of these systems, the present invention introduces a movable pin cell culture system for cell based assays. The system is not only suitable for 2D monolayer and 3D cell culture, but also is capable of simultaneously testing different type of the cells which are cultured in 2D monolayer, 3D scaffold and 3D non-scaffold. This system is more flexible, easier to handle and robust for carry out the cell based assays.

### SUMMARY OF THE INVENTION

In a first aspect, the present invention relates to a pin for use in a cell based assay device comprising: a pin body comprising a pin head and a pin tip, wherein the pin tip comprises a surface for cell seeding

In an embodiment of the pin of the present invention, the pin body comprises at least a tapered segment and the pin body forms a T-shape together with the pin head.

In an embodiment of the pin of the present invention, the pin body and pin tip have a cylindrical shape.

In an embodiment of the pin of the present invention, the pin body and pin tip have a non-round cross-cut shape.

In an embodiment of the pin of the present invention, the pin head is a stepwise pin head comprising a groove.

In an embodiment of the pin of the present invention, the surface is selected from the group consisting of a flat surface, a flat surface with a raised rim at the edge, an inward structured surface, a multi-groove surface, a multi micro-well surface, a multi micro-pillar surface, a rough surface, a permeable membrane, a porous segment or a magnetic segment; preferably the layer comprises a layer promoting cell adhesion, more preferably a cell adhesion promoting layer selected from hydrogel.

In a second aspect, the present invention relates to a pin support comprising a plurality of openings for receiving a pin according to the present invention, wherein the pin support comprises a ring-shaped part connected to a cylindrical part to form a T-shaped pin support, wherein the diameter of the ring-shaped part is bigger than the diameter of the cylindrical part, wherein the ring-shaped part comprises the plurality of openings for receiving the pins.

In an embodiment the pin support is made of plastic, preferably polytetrafluoroethylene, polystyrene, polyester and polycarbonate.

In an embodiment the pin support is sized so as to fit in the well of a multi-well plate.

In a third aspect, the present invention relates to a device to perform a cell based assay comprising:

a pin support comprising a plurality of openings for receiving a pin according to the present invention and a base comprising a groove channel

In an embodiment of the device to perform a cell based assay, the pin support and the base have a matched shape and the openings for receiving a pin are arranged such that they are aligned with the groove channel of the base.

In an embodiment of the device to perform a cell based assay, the pin support further comprises an opening for receiving a cell culture insert and the base further comprises a reservoir for receiving a liquid, wherein the reservoir is arranged within the base such that it is aligned with the opening of the pin support for receiving a cell culture insert.

In an embodiment of the device to perform a cell based assay, the device further comprises a pump which is in fluidic connection to the reservoir, to the groove channel and to the cell culture insert.

In an embodiment of the device to perform a cell based assay, the pin support comprises:

a plurality of openings for receiving a pin,
at least two openings to receive at least two cell culture inserts,
at least one opening to receive the shaft of the pump, and

the base comprises:

at least two reservoirs,
at least one opening to receive the pump, in particular a pump impeller,
an inlet channel,
an outlet channel,

wherein the inlet channel and the outlet channel of the pump are connected to the reservoir and to the groove channel

In an embodiment of the device to perform a cell based assay, the device comprises a pin support comprising a plurality of openings for receiving a pin according to the present invention, a base comprising a groove channel and a reservoir, wherein the groove channel is sized so as to receive the pin tips of the pins being secured in the openings of the pin support, wherein the groove channel is connected to the reservoir.

In an embodiment of the device to perform a cell based assay, the pin support and the base are separable parts of the device.

In an embodiment of the device to perform a cell based assay, the pin support and the base are integrated as a single part.

In an embodiment of the device to perform a cell based assay, the base comprises more than one groove channel

In an embodiment of the device to perform a cell based assay, the groove channel has a stepwise shape comprising an upper part, a step and a lower part, wherein the upper part has a bigger dimension than the lower part.

In an embodiment of the device to perform a cell based assay, the groove channel is in a linear or circular arrangement in the base.

In an embodiment of the device to perform a cell based assay, the reservoir contains the liquid.

In an embodiment of the device to perform a cell based assay, the pin support, the pins and the base are made of plastic, preferably polystyrene, polyester and polycarbonate.

In a fourth aspect, the present invention relates to a cell-seeding device for seeding cells on a pin surface of a pin as defined herein, such cell seeding device comprising:

a first part and a second part forming a plurality of funnel shaped cavities when assembled, wherein the funnel shaped cavity comprises an upper cavity part with an opening and a lower cavity part with an opening, wherein the lower cavity part of the funnel shaped cavity is sized so as to receive the tip of the pin.

In an embodiment of the cell seeding device, the funnel shaped cavities are arrayed in a matrix.

In an embodiment of the cell seeding device, the matrix is an 8-, 12-, 16, 24-96-funnel format.

In an embodiment of the cell seeding device, the lower part of the funnel shaped cavity and the inserted tip of the pin form a liquid tight seal.

In an embodiment of the cell seeding device, the cell seeding device is made of plastic, preferably polypropylene, rubber, polystyrene, polyester and polycarbonate.

In an embodiment of the cell seeding device, the funnel shaped cavity is formed as a single piece.

In an embodiment of the cell seeding device, the cell seeding device comprises a removable reduction sleeve in the upper cavity part, wherein the sleeve comprises a chamber having openings at both ends and the end of the sleeve next to the lower cavity part has an opening with a size smaller than the diameter of the pin tip.

In an embodiment of the cell seeding device, the end of the sleeve next to the lower cavity part contains multi micro-openings.

In an embodiment of the cell seeding device, the sleeve contains a separator to form multi-chambers inside the sleeve, wherein each chamber contains at least one micro-opening at the end of the sleeve next to the lower cavity part.

In an embodiment of the cell seeding device, the upper part cavity has the same diameter as the lower cavity part.

In an embodiment of the cell seeding device, the reduction sleeve is placed into the upper part cavity of the cell seeding device to allow cells to attach to the pin tip in a designed shape or pattern.

In a fifth aspect, the present invention relates to a kit for performing a cell based assay comprising:

a plurality of pins as defined herein,

at least one device to perform a cell assay according to the present invention,

at least on cell seeding device according to the present invention and

a manual to perform the cell based assay and the cell seeding.

In an embodiment of the kit, the kit further comprises the reagents to perform the cell based assay.

In a sixth aspect, the present invention relates to method for performing a cell based assay comprising:

preparing pins by attaching cells to the pin surface and culturing the cells to form 2D monolayer cells or 3D structured cell on the pin surface 6a,

filling the reservoir of the base with test medium, assemble the pin support and the base to form the device to perform a cell based assay,

inserting the cell pins into the openings of the pin support to secure the pins so as to immerge the pin surface in the test medium, and

incubating the cells in the test medium for a defined time, wherein the test medium flows in the groove channel, and after completion of the incubation analyzing substances in the test medium and/or the cells on each of the cell pins.

In an embodiment of method for performing a cell based assay of the invention, the cells are covered at the outmost surface of the pin surface by a thin layer of hydrogel to prevent cells from detaching.

In an embodiment of the method for performing a cell based assay of the invention, the cell pins are prepared from a set of target cells, wherein each of the cell pins contains one type of the target cells cultured in 2D or 3D form.

In a seventh aspect, the present invention relates to a method for seeding cells on the pin surface of a pin comprising:

inserting pins as defined herein in the opening of the lower cavity part of a cell seeding device according to the present invention to form a liquid tight seal,

adding cell suspension to each opening of the upper part cavity of the cell seeding device and culturing the cells for a defined time to allow the cells to adhere to the pin surface, and

disassembling the device and transferring the pins to a device to perform a cell based assay as defined herein.

In an embodiment of the method for seeding cells on the pin surface of a pin, the pins are moved along the lower cavity part to draw the liquid into the lower cavity part.

In an embodiment of the method for seeding cells on the pin surface of the pin, the sleeve is used for seeding a first type of cells on the surface of the pin and the sleeve is removed after the first type of cells are attached to the pin surface, and a second type of cells is added to the upper part cavity and the second type of cells are cultured to from a second cell layer on top of the cell layer of the first type of cells.

In an embodiment of the method for seeding cells on the pin surface of a pin, the pin tip contained a layer of cells prior to step a).

### BRIEF DESCRIPTION OF THE DRAWINGS

FIG. 1A, FIG. 1B, FIG. 1C and FIG. 1D show the movable pin cell culture system, with FIG. 1A showing a front view, FIG. 1B showing a side view, FIG. 1C showing a top view of the movable pin cell culture system, and FIG. 1D is a crosscut view along line AA of the base showing the reservoirs and the stepwise groove channel

FIG. 2 shows a pin placed with the pin point upward.

FIG. 3 illustrates a segment of the pins towards the pin point with inward surface.

FIG. 4A, FIG. 4B, FIG. 4C, FIG. 4D, FIG. 4E, and FIG. 4F show a multi-unit cell seeding device, with FIG. 4A showing front view; FIG. 4B showing side view; FIG. 4C showing top view of the device; FIG. 4D showing BB line crosscut view of the device showing the reservoir formed by the upper portion of the funnels; FIG. 4E showing CC line crosscut view of the device showing the pin 1 in the narrow tube of the funnel and FIG. 4F showing FF line crosscut view shows the connection portion of the upper portion of the funnels.

FIG. 5A, FIG. 5B and FIG. 5C together show a movable pin cell culture system with a circular groove channel with FIG. 5A showing front view, FIG. 5B showing top view of the system, and FIG. 5C showing DD line crosscut view of the base showing the pin 6 and the groove channel

FIG. 6A, FIG. 6B and FIG. 6C illustrate some top views of variants of groove channels which can be used in the assay system of the present invention.

FIG. 7 is a hollow pin and a removable magnetic insert with front view and top view.

FIG. 8A and FIG. 8B are examples of pins (FIG. 8A) and funnel-like (FIG. 8B) cell seeding strips.

FIG. 9 is an example of T-shaped inserts.

FIG. 10 is an example of T-shaped inserts in a 12-well plate.

FIG. 11 is an example of T-shaped inserts with pins in wells.

FIG. 12A, FIG. 12B, FIG. 12C, FIG. 12D and FIG. 12E show a reduction sleeve for the upper cavity part of a funnel shaped cavity. FIG. 12A shows a front view, FIG. 12B shows a top view with single opening, FIG. 12C shows a top view with multi-openings, FIG. 12D shows a top view with separators of the sleeve, and FIG. 12E shows a front view of the sleeve in a cell seeding device.

FIG. 13A, FIG. 13B, and FIG. 13C show an example of a movable pin cell culture system 1a with cell culture inserts and an integrated centrifugal pump. FIG. 13A shows a perspective view of the cell culture system; FIG. 13B shows a top view of the assembled system with pins and cell culture inserts; FIG. 13C shows a bottom view of the assembled system with pins and cell culture inserts.

### DETAILED DESCRIPTION OF THE INVENTION

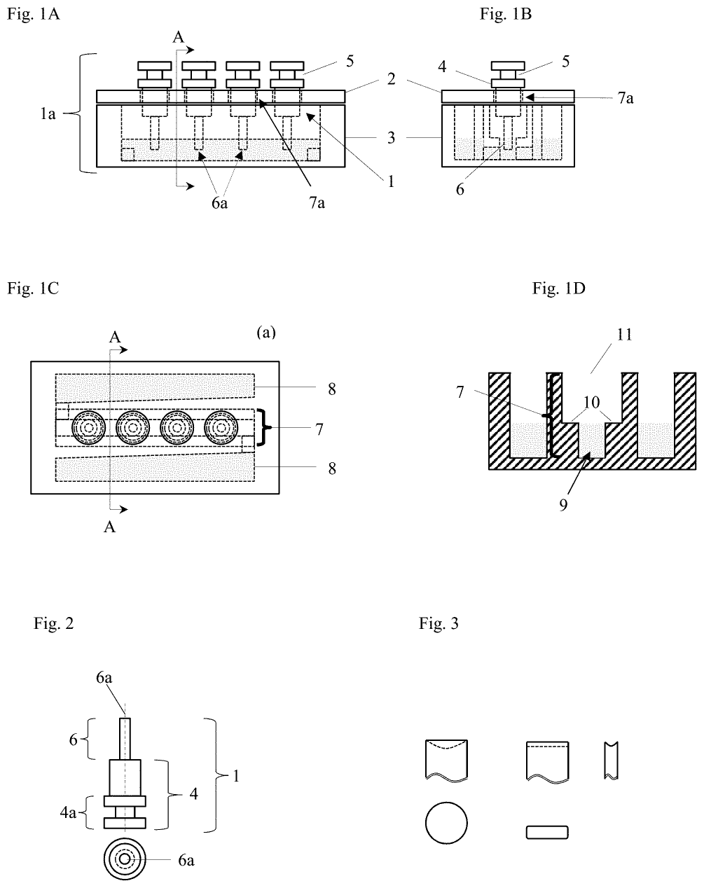

FIG. 1A, FIG. 1B, FIG. 1C and FIG. 1D depict an exemplary movable pin cell culture system 1a. The cell culture system 1a comprises individual movable pins 1, a pin support 2 and a base 3. The pin 1 has a stepwise pin head 4a with a groove 5 and a pin tip 6 of which the surface 6a is for 2D or 3D cell culture. The pin support 2 is placed on the base 3 and the pin support 2 comprising openings 7a for receiving the pins 1 so as to secure the pins 1 in the movable pin cell culture system 1a. The pin support 2 is also used as a cover for the base 3. The base 3 contains stepwise groove channels 7 and two trapezoid reservoirs 8. Each side of the channel 7 has one of the reservoirs that are connected by the bottom narrow groove 9 in an S-shaped arrangement. When in use, a test medium (red dots in the drawings) is added to one of the reservoirs 8 and the medium will flow through the narrow groove 9 to another reservoir. The pin support 2 is placed on the base 3 and then the cell pins 1 are inserted into the receptacle openings 7a on the pin support 3 by a tweezer, e.g. with a crab tip, which can hold the pin 1 at the groove 5. The stepwise pin heads 4a will be secured by the groove channels 7 so that the pin points immerge in the medium in the narrow groove 9. A rocker shaker is used to circulate the medium back-and-forth through the narrow groove 9 from one reservoir 7 to another during the incubation. The shaft axis of the seesaw movement is parallel to the channel Depending on the assay the result can accordingly be read out by analysis of the test medium and/or the cells on the cell pins 1. For example, the system is capable of testing the cell pins 1 with either 2D monolayer or 3D non-scaffold form of hepatocytes in a drug metabolism assay. The test compound and its metabolites in the medium will be analyzed after incubation. For a cell toxicity assay, a set of target cells can be used to prepare the cell pins so that they can be tested in the same environment. Therefore, the results may be more relevant to the in vivo situation. The toxic effects on the cells, e.g. changes in cell morphology, the capability of forming or accumulation of colored or fluorescent substance inside cells and so on, can be examined by using a microscope or a spectrophotometer. In some circumstances the individual cell pins can be transferred to a multi-well plate to carry out further assays.

FIG. 2 depicts an exemplary pin 1 comprising a flat pin point 6 and a stepwise round column pin head 5. The surface 6a of the flat pin tip 6 is for cell attachment and cell growth. In some cases, the surface 6a of pin 1 may contain a slightly raised rim at the edge so that it can retain more medium at the tip point to avoid drying off during the assembling of the assay system. Although various shapes of the pins 1 can be used for both 2D and 3D cell culture according to the present invention, a large round surface is preferred for preparing monolayer of cells, a narrow rectangular surface is preferred for preparing multicellular 3D cells to get better diffusion of nutrients to the center of the cell clusters, and a permeable membrane surface is preferred for preparing multi-layered large cell sheet to ensure the nutrients reaching to the deep layer of cells. Besides a flat surface, the surface 6a can be an inward structured surface e.g. concave, cone, pyramid, or half-cylindrical form (FIG. 3), a multi-groove, a multi micro-well, a multi micro-pillar surface of the pin point and a rough surface of the pin point can also be used, especially for 3D cell culture with hydrogel. In another embodiment, a pin 1 for 3D scaffold cell culture contains a porous segment towards the pin point. Although there is no special limitation on the size of the pin 1, the size of the pin surface 6a is preferably to be within a microscope field of view for easy observation. A preferred pin surface size is a circular area with a diameter from 0.1 to 2.5 mm.

The attachment of the cells direct to the surface of the pin surface 6a is performed by using a cell seeding device 40 in which the pins 1 are placed upward. So the cells in the cell suspension will precipitate and attach to the surface 6a of the pin 1 by the gravity and form a monolayer or multicellular 3D cell cluster. According to an embodiment of the present invention, the cell seeding device 40 comprises two compartments, a first part 41 and a second part 43 which both contain half-funnel like cavities. When put together the two compartments 41 and 43 form funnel shaped cavities for receiving pins 1. Each funnel shaped cavity 44 comprises an upper cavity part 44a with an opening and a lower cavity part 44b with an opening, wherein the lower cavity part 44b of the funnel shaped cavity 44 is sized so as to receive the tip 6 of the pin 1. In an embodiment of the invention, the diameter of the lower cavity part 44b preferably corresponds to the diameter of the pin tip 6. When the pins 1 are placed in the lower cavity part 44b, the two compartments 41 and 43 are tightened together to seal the gaps which prevents leakage of culture medium. In an embodiment of the invention, a gasket e.g. rubber ring or rubber sheet, may be placed around the contact surface to prevent leakage of medium. When the cell-seeding device 40 is made of elastic materials e.g. silicone a gasket can be omitted. It is preferred that the pin tip 6 does not protrude into the cavity 44 especially for the 3D cell culture. Thus, all the cells can attach to the surface 6a of the pin tip 6 to reduce the cell number variability among the pins. In order to avoid the formation of air bubbles in the lower cavity part 44b, which happens quite often for a small sized tube with one end closed, the pin 1 can be moved up and down several times to remove the trapped air. In another embodiment, the cell seeding device can also be made as a single compartment instead of two. A segment of tube is also possible to be used for seeding the cell since a suitable sized tube and pin can form a syringe like device in which the tube functions as a barrel and the pin as a piston. Therefore, the cell suspension can be drawn into the barrel. When the size of the pin is large enough, a pipette can even be used to add the cell suspension to the tube where the pin point is set below the inner opening of the tube. For 3D cell culture, the surface of the funnel like cavity is preferable to have a cell repellent layer, which can be formed by coating or grafting appropriate chemicals or polymers.

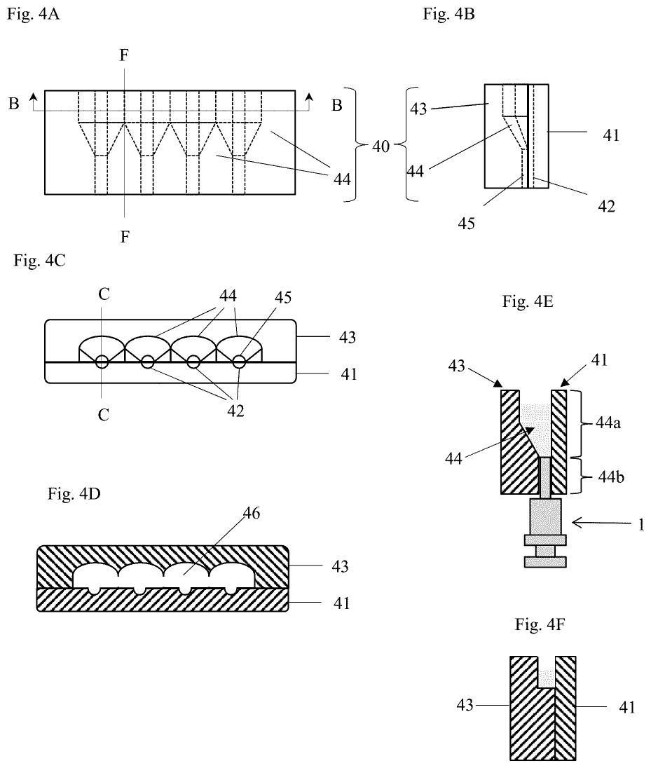

FIGS. 4A-4F depict a multi-funnel cell seeding device 40 comprising two different structured cavity compartments. One compartment 41 has a number of half-round channels 42 and the second compartment 43 has a matching number of half-funnel structured cavities 44 in which the upper wide part of funnels are connected (FIG. 4F). When assembled, the half-round channels 42 and the corresponding narrow half-tubes 45 of the funnels 44 form a narrow tube for accommodation of the pin 1 (FIG. 4E). The connected upper wide part of the funnels becomes a reservoir 46 (FIG. 4D). Therefore, cell suspension can be poured to the reservoir 46. Once its level goes up into the reservoir the cell suspension can freely flow at the upper part of the funnels. After placing the multi-funnel quick cell seeding device 40 horizontally, the cells will evenly be distributed to the funnels and precipitate on the surface 6a of the pin tip 6 by the gravity. Each of the pins 1 will receive the same amount of cells for growth. The growing status of the cells can be observed from the thin compartment of the half-round channel The use of the multi-funnel quick cell seeding device 40 not only eases the handling, but also reduces the variation caused by many times of pipetting to transfer cell suspension to each of the funnels since the cell suspension quickly becomes uneven due to the quick precipitation of the cells.

The principle of multi-funnel cell seeding device can also be applied to make a cell-repellent surface multi-well plate such as e.g. a 96- or 384- or 1536 well plate for the preparation of spheroids, in which the wells of the multi-well plate are square wells with conical or round bottom. All the square wells open to a reservoir that is formed by a raised bank around the plate. There is a groove between the bank and the outmost wells to minimize uneven distribution of the cells caused by liquid rising around the bank. The upper portions of the wall between the wells and the groove are connected to each other forming an angular rooftop structure to prevent cell landing and to equally guide cells to the adjacent wells. Thus the cells can seamlessly be distributed to the wells when the cell suspension is added to the reservoir with a volume enough to cover the openings of the wells. Once the cells precipitate to the bottom of each well, the culture medium in the reservoir can be removed and subsequently it can be handled just like a normal multi-well plate. The cells will form a single spheroid in each well after cultured in an incubator.

Materials non-toxic to cells can be used to make the movable pin cell culture system 1a and the cell seeding device 40. Plastics such as polystyrene, polyester, polycarbonate, polypropylene and polyoxymethylene are preferred. Due to the various conditions of cell attachment, the surface 6a of the pin point 6 may need to be treated by different means such as e.g. gas plasma treatment, polymer grafting, and extracellular matrix coating. Polytetrafluoroethylene may be used to make the cell seeding device 40 as well as the pins 1 to avoid the leakage of the medium and the generation of air bubbles as well. A narrow hydrophobic gap between the pin 1 and the narrow tube can function as a selective barrier because it allows air to pass through freely but prevents the medium from leakage. The hydrophobic gap can also be made by coating of a hydrophobic material on the contact surface of the pin and the cell seeding device.

With the cell seeding device 40, single cell suspension, biopsy micro-tissues and 3D cell culture produced organoids/spheroids can be seeded directly to the pin surface 6a. Once the cells tightly attach to the surface 6a of the pin tip 6, the pins 1 can be transferred to a multi-well cell culture plate for growth. For cells from e.g. lung cancer cell lines, some of the surface cells will break off the cell cluster during the cell culture since the cell pin 1 is placed downward in the multi-well plate. In order to prevent shedding off of surface cells, the attached cells can be covered by a thin layer of hydrogel. Suitable hydrogels are agarose, alginate and collagen. The thin hydrogel coating layer can be formed by first dipping the pin tip 6 into the hydrogel solution and then dipping the pin tip 6 into an appropriate solution to solidify the hydrogel. For example, agarose can be solidified by a cold medium. An alginate solution can be solidified with a BaCl₂ or CaCl₂ solution, and collagen can be solidified by a warm alkaline solution.

Besides a thin hydrogel coating layer, a pin 1 containing a magnetic segment at the pin tip 6 can also be used to confine the cells to the pin tip surface 6a when the cells are treated with paramagnetic nano-particles to become magnetized cells or cell clusters during the cell culture on the pin. FIG. 7 is an example of a hollow pin 71 with a removable magnetic insert 72 which contains a piece of a magnetic block 73 at the tip. The head of the pin has a groove 74 shaped to accommodate the head 75 of the insert. When the insert is placed in the hollow pin, a magnetic pin is formed which can attract ferromagnetic and paramagnetic materials at the pin point 76. The magnetic pin can also be made by embedding a magnetic block to the pin point, in which the magnetic block can be either hard or soft magnetic materials. A soft magnetic material is preferred since it can have both magnetization and demagnetization status. When the cells are magnetized directly on the tip point to introduce a demagnetization washing step is preferable in order to remove the unbound paramagnetic nano-particles. In addition, by using magnetized cells the magnetic pin can directly immobilize the cells to the pin point from the cell suspension. Therefore, the applications of the movable pin cell culture system can be expended to non-adherent cell assay, polarity cell assay with a defined apical-basal polarity orientation by using the paramagnetic nano-particles that specifically bind to either apical or basolateral membrane, and an assay where the target cells are directly fished out from a cell mixture with several cell types by using specific paramagnetic nano-particles to the target cells. Besides, the applications can further be widened to magnetic bead immunoassays, especially multiplex assays in which different targeting beads can be reacted with a sample solution one-by-one due to easy and completely transferring of beads by the magnetic pins.

The cell seeding device 40 can also be used for the preparation of multi-spheroids with hydrogel which requires long time incubation for solidification. For example, to prepare cells in a low concentration of collagen solution, a tiny amount of cells suspended in the collagen solution is added to the surface 6a of the pin tip 6 in the cell seeding device 40 and the cell solution is covered by a lower density alkaline solution with an appropriate pH. After incubation at 37° C. for certain time, the collagen solution will slowly form a hydrogel that sticks on the surface 6a of the pin tip 6 with the embedded cells. Each cell in the hydrogel will grow to form a spheroid.

According to an embodiment of the present invention, the movable pin cell culture system 1a uses a stepwise groove channel 7 to minimize the volume change of the culture medium in the channel during the seesaw movement by a rocker shaker (FIG. 1D). In the stepwise groove channel the step 10 is to prevent the medium from climbing into the upper wide groove 11. Whereas, the capillary force of the narrow groove 9 will keep the medium in the narrow groove to ensure that the cells on the surface 6a of the pin tip 6 can always be in the medium. It is preferable for the narrow groove to have a gap size so that the capillary force of such a narrow groove is able to raise the medium to a height higher than the step. Therefore, by introducing the step below the maximum raising height to stop the medium climbing up further, the stepwise groove channel can retain the same volume.

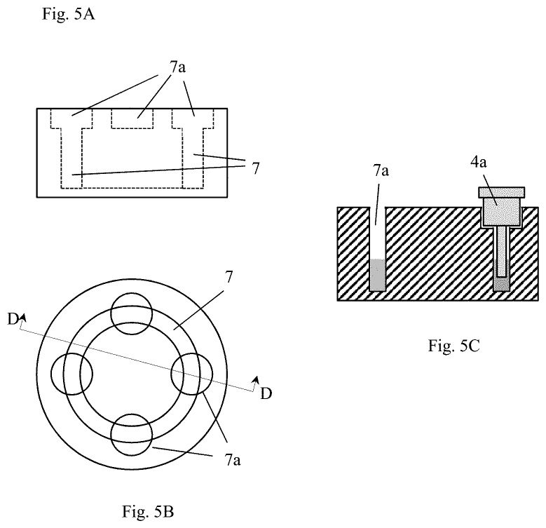

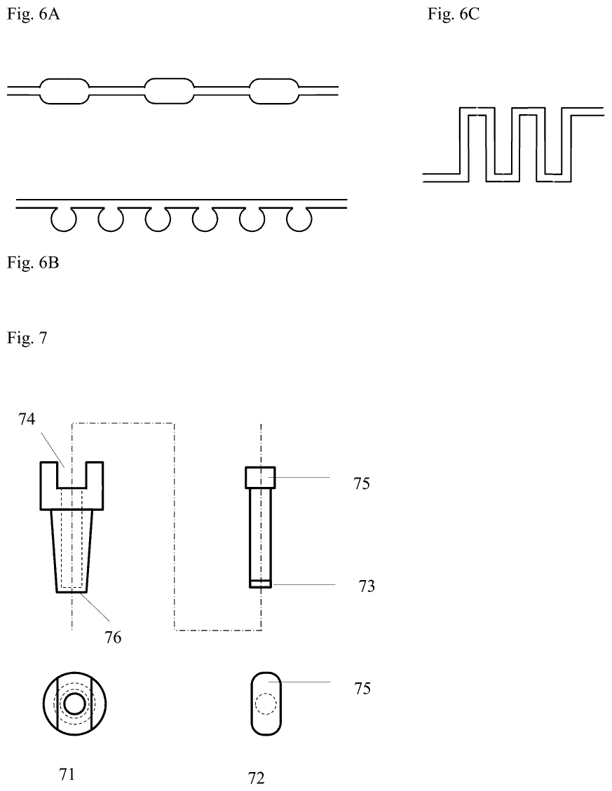

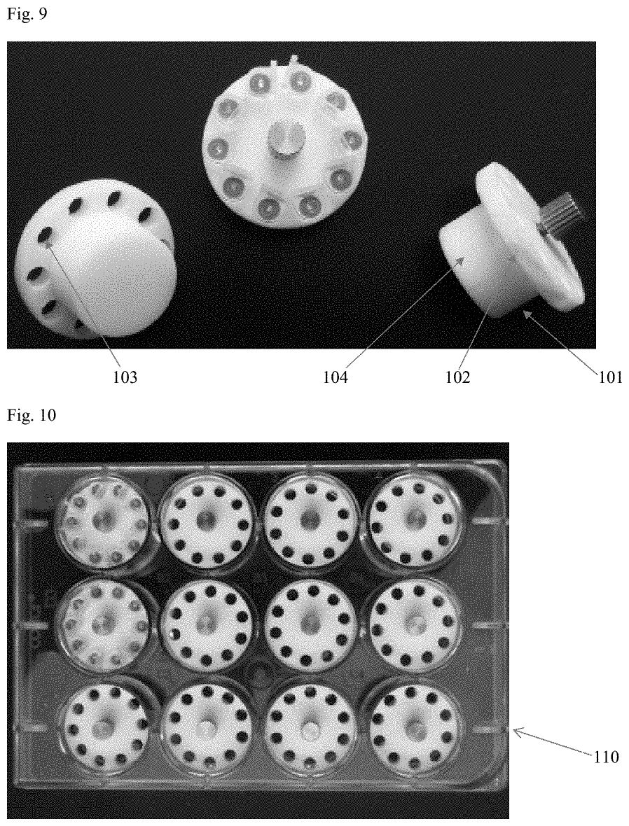

In another embodiment, the base of the movable pin cell culture system (FIGS. 5A-5C) contains a base with a circular groove channel 7 with receptacle openings 7a for the cell pins 6. The receptacle openings 7a are located at the upper part of the channel 7. When in use, the pins 1 are inserted one-by-one along the circular groove channel 7 and the T-shaped pin heads 4a will be secured in the receptacle openings 7a so that the cells attached to the surface 6a of the pin tip 6 are immerged in the medium. A vertical and horizontal orbital motion waver shaker can be used to drive the culture medium flowing clockwise or counterclockwise in the groove channel 7. The movable pin cell culture system with a circular channel is preferred for assays requiring high ratio of cell numbers to the incubation volume. The circular channel 7a can also be made by another method in which the channel 7 is formed by placing a T-shaped insert 101 in a matching well of a multi well plate (see FIG. 9 and FIG. 10). The top protruding structure of the T-shaped insert 102 contains holes 103 for the cell pins 1 and its size is preferably the same as that of the well so the T-shaped insert 101 can be secured in the middle of the well and the circular groove channel 7a is formed. In some instances, a stepwise groove channel may be preferred when cells are sensitive to the flow of the medium. In this case, the step is covered by the medium so the upper wide groove also serves as a flow channel to decrease the flow rate of the medium in the narrow groove so that the cells on the surface 6a of the pin tip 6 will be less affected. Several variants of groove channels are shown in FIGS. 6A-6C. For example, the gap size of the groove channel may be different along the groove channel (FIG. 6A) so as to fit both small sized pins 1 and large sized pins 1 when an assay requires different amount of cells from several cell types. A design of wells along a narrow channel (FIG. 6B) can minimize the total volume when large sized pins 1 are used. When the bottom of the wells is lower than that of the narrow channel, the wells can also be used for cell culture. Thus, combined with the pins 1, the ratio of cells to medium can further be increased. A groove arranged in the snake shape (FIG. 6C) can accommodate more cell pins but without increasing the length of the base.

The moveable pin assay system 1a can be applied not only to the groove channel 7, but also to a tube channel where the receptacle openings are arranged along the tube. A screw and nut structure is used to fasten the pins to the receptacle openings for liquid tight seal. The circulation of the medium in the tube can be driven by a tubing pump. Due to the enclosed space of the tube, it is not necessary for the pin 1 to be installed with the pin point downward. Since all directions around the tube can be used to install the cell pins 1, the cell amount can dramatically be increased in the moveable pin assay system 1a and it can fit assays requiring high cell number to volume ratio.

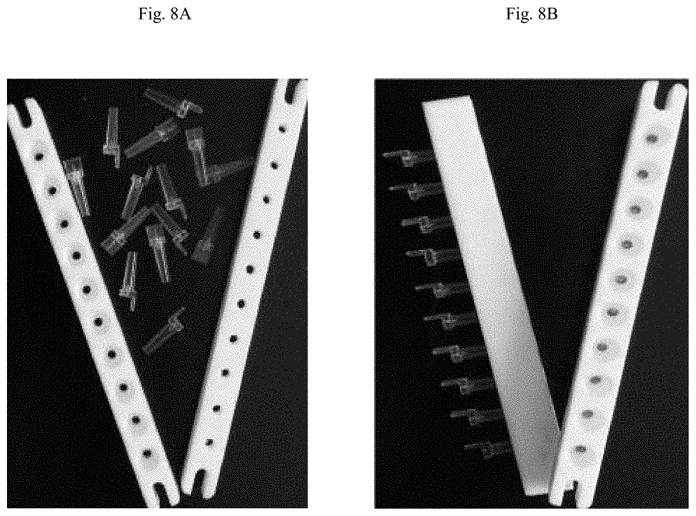

FIG. 8A and FIG. 8B depict pins 1 and corresponding cell seeding devices 40.

FIG. 9 depicts an exemplary pin support 101 with a plurality of openings 103 for receiving a pin 1 according to the present invention. The pin support 101 comprises a ring-shaped part 102 connected to a cylindrical part 104 to form a T-shaped insert 101. The diameter of the ring-shaped part 102 is bigger than the diameter of the cylindrical part 104 and the ring-shaped part 102 comprises the plurality of openings 103 for receiving the pins 1. The pin support 101 is sized so as to fit in a well of a multiwell plate.

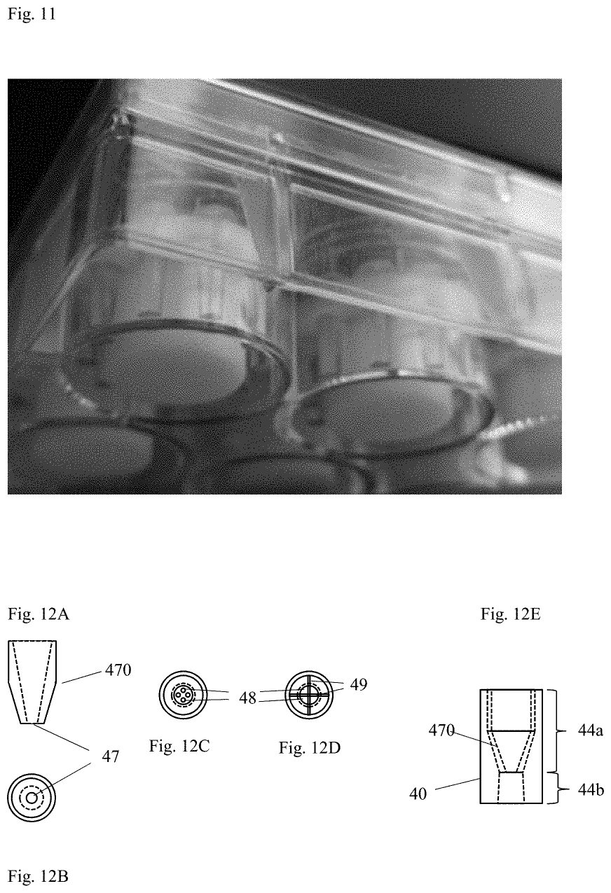

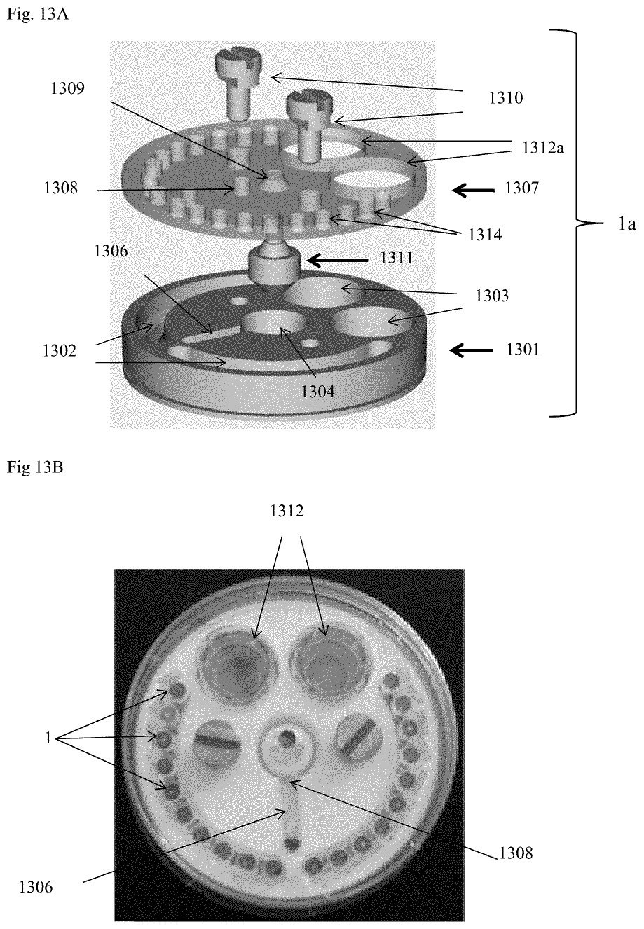

FIG. 10 depicts a multiwell plate 110 with inserted pin supports 101. Such an assembly is a functional system to perform a cell based assay according to the present invention. An exemplary cell based assay using such an assembly according to the present inventions comprises the following steps: the wells of the multiwell plate are filled with cell culture medium and in each well a different test compound is added. Then the pin supports 101 are put in the wells of the multiwell plate 110 and pins 1 with seeded cells on their pin tip surface 6a are inserted in the openings 103. The cells seeded on pin tip surface 6a are in contact with the cell medium containing the test compound. The pin tip surfaces 6a can be seeded with the same cell type or different cell types. The multi well plate 110 is placed in an incubator for a defined time and the cell medium and/or the cells seeded on the pin tip surface 6a are analyzed after completion of the incubation. FIG. 11 shows a bottom view of the multiwell assembly shown in FIG. 10.

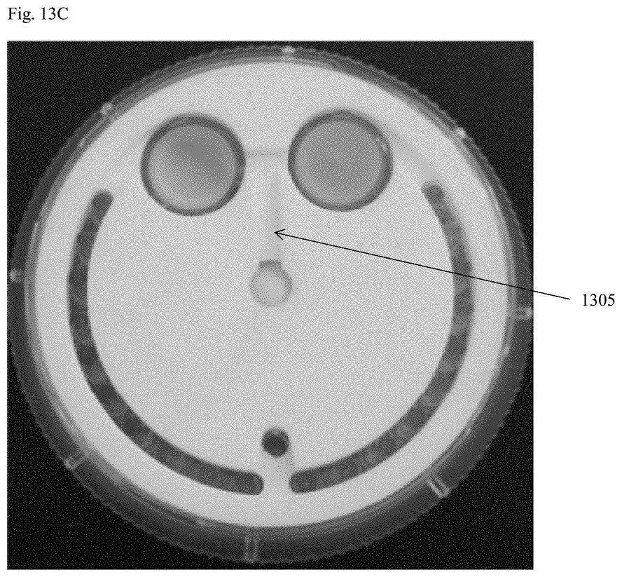

FIGS. 13A-13C show another embodiment of a device to perform a cell based assay (1a), which integrates a centrifugal pump 1311 which can be driven by a magnetic mixer. The device to perform a cell based assay 1a comprises a base 1301, a magnetic driven pump 1311 and a pin support 1307. The base 1301 has a groove channel 1302, two reservoirs 1303 to receive two cell culture inserts 1312, and an opening 1304 to receive the pump 1311, in particular a centrifugal pump impeller 1311, which are connected by pump inlet channel 1305 and pump outlet channel 1306 so as to allow a liquid, for example a cell culture medium, to circulate through these cavities when assembled. The pin support 1307 comprises openings for cell culture inserts 1312 and openings 1314 for receiving pins 1, as well as sampling opening 1308 and pump shaft bearing opening 1309. The screws 1310 serve to fix the base 1301 to the pin support 1307.

Before use, the cell based assay system 1a comprising the pin support 1307 and the base 1301 are assembled. The assembly of the cell based assay system 1a comprises the following steps:

1) putting the base 1301 into a petri dish,

2) placing the pump impeller 1311 in the pump house 1304,

3) putting the pin support 1307 on the base and

4) fastening the screws 1310 to fasten the base 1301 to the pin support 1307.

Then, the cell culture medium is filled in the assembled cell based assay system, and the cell culture inserts 1312 and the cell pins 1 are placed to their correspondent positions and the assembled system is covered with a petri dish lid. Afterwards place the system on a magnetic mixer in a cell culture incubator. Set a proper rotation speed of the mixer and the impeller 1311 will turn at a speed accordingly so as to drive the culture medium flowing through the cavities with a defined flow rate. The sampling opening 1308 can be used for addition of compounds as well as sampling the test medium for analysis. Since the rotation direction of the mixers may be different from different suppliers a bi-direction impeller, such as bladeless impeller, is used in the system.

Cell culture inserts are permeable supports which serve as tools for the study of anchorage dependent and independent cell lines. Cell culture inserts comprise a membrane on which cells can be grown. A preferred cell culture insert is a Transwell® cell culture insert. Transwell® cell culture inserts are convenient, sterile, easy-to-use permeable support devices for the study of both anchorage-dependent and anchorage-independent cell lines. Transwells® are commercially available from cell culture device manufacturers such as e.g. Fisher Scientific or Sigma Aldrich.

In a further embodiment, the movable pin cell culture system 1a may also comprise receptacle openings for a cell culture insert 1312a. The receptacle openings for the cell culture insert 1312 are preferably located above the reservoir 1303. The cell culture inserts 1312 can be used to prepare cell sheets and function as a barrier for chemical compounds. Therefore, a more complex assay can be performed to simultaneously studying adsorption, metabolism and secretion of a chemical compound. For example, the assembled system 1a contains an intestine cell sheet insert 1312 in one reservoir 1303, a kidney cell sheet in another cell sheet insert 1312 and hepatocyte cells seeded on the surface 6a of the pins 1 in the channel 1302. When a test compound is added to the inner chamber of the intestine cell sheet insert 1312, the intestine cell will absorb the compound and transport to the channel 1302. The hepatocytes seeded on the surface 6a of the pins 1 metabolize the compound and then the kidney cells excrete the compounds into the inner chamber of the kidney cell sheet insert 1312. A useful data can be obtained through the analysis of the compound and metabolites in the medium from the different compartments.

### EXAMPLE: USE OF THE CELL BASED SYSTEM IN DRUG METABOLISM STUDIES

Step 1: Coat the pins 1 with collagen at 0.02 mg/mL for 3 hours and dry the pins at room temperature overnight.

Step 2: Seed the hepatocytes at 0.8-1 million/mL concentration using the seeding device of the present invention, remove the pins after overnight incubation and place the pins in the pin support 101 as shown in FIG. 9.

Step 3: Place the pin support 101 with the cell coated pins 1 in an upside down position in a 12 well cell culture plate which contains 0.8-0.9 mL of cell culture medium and a test compound (see FIG. 10).

Step 3: Place the 12 well cell culture plate on a rock shaker set to shake at a speed of 10 rpm in cell culture incubator.

Step 4: Sampling at defined time points by transferring 20 μL of the culture medium into a well of a plate which contains acetonitrile.

Step 5: Analyze the samples by using LC-MS/MS for drug metabolites.

1. A pin (1) for use in a cell based assay device (1a) comprising a pin body (4) comprising a pin head (4a) and a pin tip (6), wherein the pin tip (6) comprises a surface (6a) for cell seeding.

2. The pin of claim 1, wherein the pin body (4) comprises at least a tapered segment and the pin body (4) forms a T-shape together with the pin head (4a).

3. The pin of claim 1, wherein the pin body (4) and pin tip (6) have a cylindrical shape.

4. The pin of claim 1, wherein the pin body (4) and pin tip (6) have a non-round cross-cut shape.

5. The pin of claim 1, wherein the pin head (4a) is a stepwise pin head comprising a groove (5).

6. The pin of claim 1, wherein the surface (6a) is a flat surface, a flat surface with a raised rim at the edge, an inward structured surface, a multi-groove surface, a multi micro-well surface, a multi micro-pillar surface, a rough surface, a permeable membrane, a porous segment or a magnetic segment.

7. A pin support (101) comprising a plurality of openings (103) for receiving a pin (1) according to claim 1, wherein the pin support (101) comprises a ring-shaped part (102) connected to a cylindrical part (104) to form a T-shaped pin support (101), wherein the diameter of the ring-shaped part (102) is bigger than the diameter of the cylindrical part (104), wherein the ring-shaped part (102) comprises the plurality of openings (103) for receiving the pins (1).

8. The pin support (101) of claim 7, wherein the pin support (101) is made of plastic.

9. The pin support (101) of claim 7, wherein the pin support (101) is sized so as to fit in the well of a multi-well plate.

10. A device to perform a cell based assay (1a) comprising a pin support (2, 1307) comprising a plurality of openings (7a,1314) for receiving a pin (1) according to and a base (3,1301) comprising a groove channel (7,1302).

11. The device to perform a cell based assay (1a) of claim 10, wherein the pin support (2,1307) and the base (3,1301) have a matched shape and the openings (7a,1314) for receiving a pin (1) are arranged such that they are aligned with the groove channel (7,1302) of the base (3, 1301).

12. The device to perform a cell based assay (1a) of claim 11, wherein the pin support (1307) further comprises an opening (1312a) for receiving a cell culture insert (1312) and the base (1301) further comprises a reservoir for receiving a liquid (1303), wherein the reservoir (1303) is arranged within the base (1301) such that it is aligned with the opening (1312a) of the pin support (1307) for receiving a cell culture insert (1312).

13. The device to perform a cell based assay (1a) of claim 10, wherein the device (1a) further comprises a pump (1311) which is in fluidic connection to the reservoir (1303), to the groove channel (1302) and to the cell culture insert (1312).

14. The device to perform a cell based assay (1a) of claim 10, wherein the pin support (1307) comprises:
a plurality of openings (1314) for receiving a pin (1),
at least two openings (1312a) to receive at least two cell culture inserts (1312),
at least one opening (1309) to receive the impeller shaft of the pump (1313), and the base (3,1301) comprises:
at least two reservoirs (1303) to receive a liquid, in particular a cell culture medium,
at least one opening (1304) to receive the pump (1311), in particular a pump impeller (1311),
an inlet channel (1305),
an outlet channel (1306),
wherein the inlet channel (1305) and the outlet channel (1306) of the pump (1313) are connected to the reservoir (1303) and the groove channel (1302).

15. The device to perform a cell based assay (1a) of claim 10 comprising a pin support (2) comprising a plurality of openings (7a) for receiving the pin (1) claim 1, a base (3) comprising a groove channel (7) and a reservoir (8), wherein the groove channel (7) is sized so as to receive the pin tips (6) of the pins (1) being secured in the openings (7a) of the pin support (2), wherein the groove channel (7) is connected to the reservoir (8).

16. The device to perform the cell based assay (1a) of claim 15, wherein the pin support (2) and the base (3) are separable parts of the device.

17. The device to perform the cell based assay (1a) of claim 15, wherein the pin support (2) and the base (3) are integrated as a single part.

18. The device to perform the cell based assay (1a) of claim 15, wherein the base (2) comprises more than one groove channel (7).

19. The device to perform the cell based assay (1a) of claim 15, wherein the groove channel (7) has a stepwise shape comprising an upper part (11), a step (10) and a lower part (9), wherein the upper part (11) has a bigger dimension than the lower part (9).

20. The device to perform the cell based assay (1a) of claim 15, wherein the groove channel (7) is in a linear or circular arrangement in the base (3).

21. The device to perform the cell based assay (1a) of claims 15, wherein the reservoir (8) contains a liquid.

22. The device to perform the cell based assay (1a) of wherein the pin support (2), the pins (1) and the base (3) are made of plastic.

23. A cell-seeding device (40) for seeding cells on a pin surface (6a) of the pin (1) claim 1, comprising a first part (41) and a second part (43) forming a plurality of funnel shaped cavities (44) when assembled, wherein the funnel shaped cavity (44) comprises an upper cavity part (44a) with an opening and a lower cavity part (44b) with an opening, wherein the lower cavity part (44b) of the funnel shaped cavities (44) is sized so as to receive the tip (6) of the pin (1).

24. The cell seeding device (40) of claim 23, wherein the funnel shaped cavities (44) are arrayed in a matrix.

25. The cell seeding device (40) of claim 23, wherein the matrix is an 8-funnel format, 12-funnel format, 16-funnel format, 24-funnel format, or 96-funnel format.

26. The cell seeding device (40) of claim 23, wherein the lower part of the funnel shaped cavity (44b) and the inserted tip (6) of the pin (1) form a liquid tight seal.

27. The cell seeding device (40) of, wherein the cell seeding device (40) is made of plastic. 28 (Currently amended) The cell seeding device (40) of claim 23, wherein the funnel shaped cavity (44) is formed as a single piece.

29. The cell seeding device (40) of claim 23, wherein the cell seeding device (40) comprises a removable reduction sleeve (470) in the upper cavity part (44a), wherein the sleeve (470) comprises a chamber having openings at both ends and the end of the sleeve next to the lower cavity part (44b) has the opening (47) with a size smaller than the diameter of the pin tip 1.

30. The cell seeding device (40) of claim 23, wherein the end of the sleeve next to the lower cavity part (44b) contains multi micro-openings (48).

31. The cell seeding device (40) of claim 30, wherein the sleeve (470) contains a separator (49) to form multi-chambers inside the sleeve, wherein each chamber contains at least one micro-opening (48) at the end of the sleeve next to the lower cavity part (44b).

32. The cell seeding device (40) of claim 30, wherein the upper part cavity (44a) has the same diameter as the lower cavity part (44b).

33. The cell seeding device (40) of claim 30, wherein a reduction sleeve (470) is placed into the upper part cavity (44a) of the cell seeding device (40) for allowing cells to attach to the pin tip 6 in a designed shape or pattern

34. A kit for performing a cell based assay comprising:
a plurality of the pins (1) of claim 1,
at least one of said device to perform the cell based assay (1a) of claim 10,
at least one of said cell seeding device (40) of claim 23 and
a manual to perform the cell based assay and the cell seeding.

35. The kit for performing the cell based assay of claim 34, further comprising the reagents to perform the cell based assay.

36. A method for performing a cell based assay comprising:
a) preparing pins (1) by attaching cells to the pin surface (6a) and culturing the cells to form 2D monolayer cells or 3D structured cell on the pin surface (6a),
b) preparing the assay device (1a) by filling the reservoir (8,1303) of the base (3,1301) with test medium, assembling the pin support (2,1307) and the base (3,1301), and inserting the cell pins (1) into the openings of the pin support (2,1314) to secure the pins (1) so as to immerge the pin surface (6a) in the test medium, and
c) incubating the cells in the test medium for a defined time, wherein the test medium flows in the groove channel (7,1302), and analyzing substances in the test medium and/or the cells on each of the cell pins (1).

37. The method of claim 36, wherein the cells are covered at the outmost surface of the pin surface (6a) by a thin layer of hydrogel.

38. The method of claim 36, wherein the cell pins (1) are prepared from a set of target cells, wherein each of the cell pins (1) contains one type of the target cells cultured in 2D or 3D form.

39. A method for seeding cells on pin surface (6a) of a pin (1) comprising:
a) inserting the pins (1) of claim 1 in the opening of the lower cavity part (44b) of the cell seeding device (40) claim 23 to form a liquid tight seal,
b) pipetting cell suspension to each opening of the upper part cavity (44a) of the cell seeding device (40) and culturing the cells for a defined time to allow the cells to adhere to the pin surface (6a)
c).

40. The method for seeding cells on pin surface (6a) of a pin (1) of claim 39, wherein the pins (1) are moved along the lower cavity part (44b) to draw a liquid into at least the lower cavity part (44b).

41. The method for seeding cells on pin surface (6a) of the pin (1) of claim 39, wherein the sleeve (470) is removed after the cells are attached to the pin point, and a second type of cells is added to the upper part cavity (44a) and culturing the cells to from a top cell layer of the second type of cells.

42. The method for seeding cells claim 39, wherein the pin tip 6 contains a layer of cells prior to step a).
